(19) United States
(12) Patent Application Publication
(10) Pub. No.: US 2008/0242725 A1
(43) Pub. Date: Oct. 2, 2008

(54) Pharmaceutical Composition

(76) Inventors: Janez Kerc, Ljubljana (SI); Peter Svete, Borovnica (SI); Zdenka Jerala-Strukelj, Mavcice (SI); Vlasta Humar, Stahovica (SI); Rok Grahek, Kranj (SI); Breda Husu-Kovacevic, Ljubljana (SE)

Correspondence Address: SANDOZ INC, 506 CARNEFIE CENTER, PRINCETON, NJ, 08540, US

(21) Appl. No.: 10/590,780

(22) PCT Filed: Feb. 28, 2005
(86) PCT No.: PCT/EP2005/002107

§ 371 Date: Oct. 10, 2006

(30) Foreign Application Priority Data
Mar. 1, 2004 (SI) P200400067
Jun. 24, 2004 (SI) P200400186

(51) Int. Cl. A61K 31/225; A61K 47/26; A61P 9/10

(52) U.S. Cl. 514/548; 514/781

(57) ABSTRACT

Stabilized pharmaceutical compositions comprising polymorphs of active pharmaceutical ingredients susceptible to conversion to alternate polymorph forms are prepared by a process of wet granulation in which the ratios of active, fillers, and granulating liquid are controlled in order to avoid polymorphic interconversions.

### FIELD OF THE INVENTION

The present invention relates to a new method of stabilization of pharmaceutical active ingredients, particularly within pharmaceutical formulations, to prevent degradation and/or conversion of one polymorph form into other polymorph forms. Particularly the present invention relates to compositions prepared by such a method comprising the active in a desired crystalline form, which exhibits an X-ray diffraction pattern preferably with narrow peaks,

### BACKGROUND OF THE INVENTION

Existence of different polymorph forms is known in many classes of active pharmaceutical ingredients, among them: candesartan, irbesartan, telmisartan, losartan, atorvastatin and pravastatin. Polymorphs are forms of the same substance with different space (crystal packing) arrangements which can have different levels of order, i.e., crystallinity, where lower crystallinity causes peaks to broaden on an X-ray diffractogram. The ultimate form of non-order of a solid is the amorphous state, which does not show the repeatability of molecular directions and positions in a solid. Completely amorphous substance thus shows a diffuse dispersion of X-ray radiation, which is substantially manifested in a continuum of diffractions throughout the whole of the measured range. Polymorphs can be metastable, that is not in an equilibrium state of a material with respect to some transition, conversion, or reaction, but stabilized kinetically. Suitable methods for characterization of polymorphs rely for example on thermochemical analysis such as RTG or DCS or X-ray diffraction spectroscopy (“XRPD”). The diffraction pattern of scattering of X-rays from a crystal as measured by X-ray diffraction spectroscopy depends on the “long-range” order in the crystal. It is believed that narrow peaks throughout the scale up to above 30° 2θ correspond to the long range orderly crystalline structure while intense peaks at the low 2θ values i.e. up to 10° 2θ correspond to short range order.

For instance, crystalline pravastatin sodium is disclosed in U.S. Pat. No. 6,740,775 (“Form LEK”), and different forms of pravastatin sodium are disclosed in WO 01/43723. Both publications are in their entirety hereby included by reference. It is known that the pravastatin sodium forms D and H as named in WO 01/43723 convert to forms A, H, H1, I, J, K as named in WO 01/43723 by treating with alcohol. It is also stated in that patent specification that any form, except B or D would transform into form D by exposing to 120° C. Form D is characterized by three broad peaks between about 2° and 12° 2θ and one very broad peak extending from about 15° to 25° 2θ in its X-ray powder diffraction pattern, i.e. it has undesirably poor crystalline order.

Generally it is desirable to incorporate into a composition an active ingredient with an improved crystallinity i.e. having an orderly structure of significant range, which can be characterized by an X-Ray diffraction pattern exhibiting narrow peaks, that is: having half-value widths below 2°, preferably below 1°, most preferably below 0.5° 2θ. Better crystallinity may lead to improved solubility and/or ease of processing the form into pharmaceutical dosage forms, due to factors such as particle size, density and tendency of a powdered or granulated form to flow and the surface properties that determine whether particles will adhere to each other when compacted into a tablet.

However it is sometimes advantageous to incorporate into a composition an active ingredient in amorphous form which is stabilized against crystallization, for example when the solubility and bioavailability of the crystallized substance is much lower than that of amorphous. Moreover it is important to avoid any polymorphic transition which may occur during the manufacturing or the solid dosage form and especially during the storage.

Susceptibility to conversion into one or more other polymorph forms is a phenomenon whereby an unprotected substance in a first polymorph form will at least partially convert into at least one other polymorph form when exposed to adverse environmental influences. The harmful influences can be external (such as humidity, temperature) or internal, caused in the pharmaceutical composition by the interaction of the inactive ingredients (“excipients”) with the active.

### BRIEF DESCRIPTION OF THE DRAWINGS

FIG. 1 is a characteristic powder X-ray diffraction pattern of crystalline pravastatin sodium with significant peaks having half-value widths below 2° 2 Theta and which corresponds to the FIG. 2 of the U.S. Pat. No. 6,740,775 (“form LEK”)

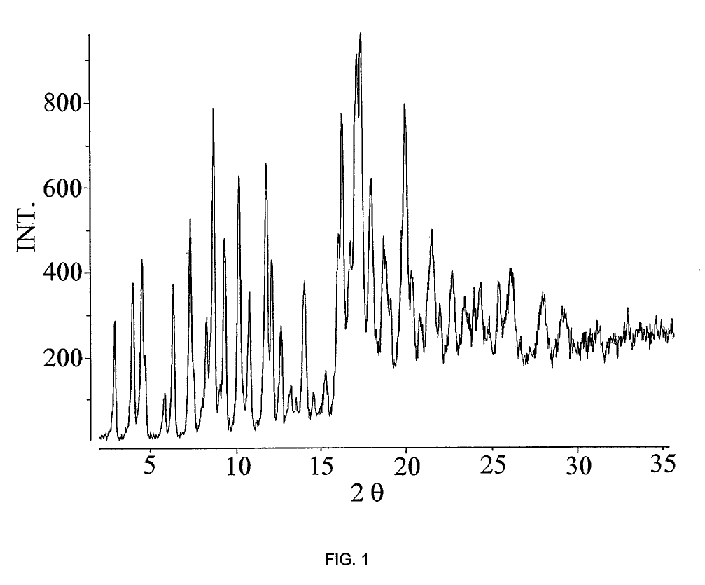

FIG. 2 is DSC thermogram of pravastatin sodium form LEK

FIG. 3 is a characteristic powder X-ray diffraction pattern of pravastatin sodium form D as named in WO 0143723 and corresponds to FIG. 7 of WO 0143723

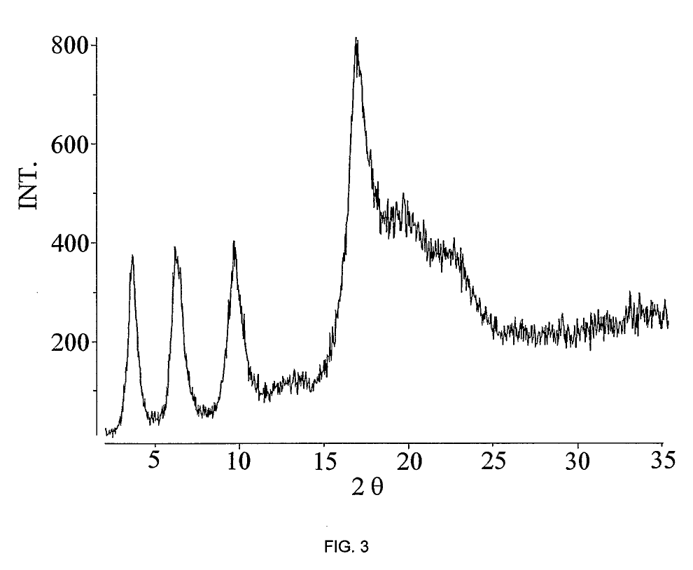

FIG. 4 is DSC thermogram of pravastatin sodium form D as named in WO 0143723

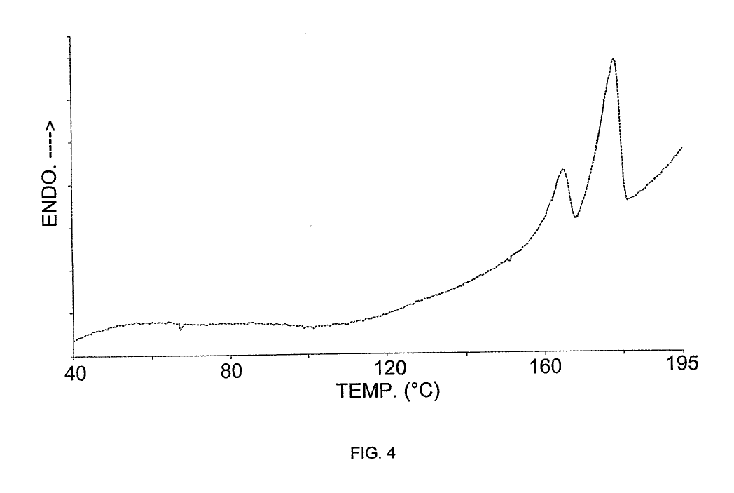

### SUMMARY OF THE INVENTION

In a first aspect, the invention provides a process for the preparation of a pharmaceutical composition comprising an active pharmaceutical ingredient capable of existing in multiple polymorphic forms, comprising a step of preparation of a wet phase comprising said active pharmaceutical ingredient and microcrystalline cellulose and liquid, wherein in said wet phase the weight ratio of active pharmaceutical ingredient to microcrystalline cellulose is above 1.0 and/or the weight ratio of active pharmaceutical ingredient to liquid is above 1.0.

In another aspect the invention provides a pharmaceutical composition obtainable by the process as described above.

In further aspect the invention provides for a use of a pharmaceutical composition as described above for the manufacture of a medicament for treatment of hypercholesterolemia and a method of preventing or treating hypercholesterolemia in a susceptible patient, comprising administering to said patient a therapeutically effective amount of the pharmaceutical composition as described above

In a specific aspect the invention is a stabilized pharmaceutical composition comprising the polymorph form of pravastatin sodium which exhibits X-Ray diffraction pattern with significant peaks having half-value widths below 2° 2 Theta characterized in that the polymorph form of pravastatin sodium is stabilized against converting into one exhibiting peaks in X-ray diffraction pattern, having half-value widths of significant peaks above 2° 2 Theta

### DETAILED DESCRIPTION OF THE INVENTION

A new method of stabilization of a pharmaceutical active ingredient, particularly one capable of existing in multiple polymorphic forms which is susceptible to polymorphic conversion against harmful environmental influences, that means a substance which exists in a first polymorph form and can convert into one or more other polymorph forms of the same substance, has been developed. The method can be applied to any active and in particular to crystalline actives, especially to actives in a polymorph form characterized by an X-ray diffraction pattern exhibiting narrow peaks. Particularly the stabilization method can be applied to HMG CoA reductase inhibitors, which are known to exhibit polymorphism, such as atorvastatin and its salts, preferably to pravastatin sodium. Pravastatin sodium can be used in tablet form for treating hypercholesterolemia, i.e. reduction in serum cholesterol levels by administration of a solid dosage form of pravastatin sodium, for instance in daily dosages of 10, 20, 40 or 80 mg.

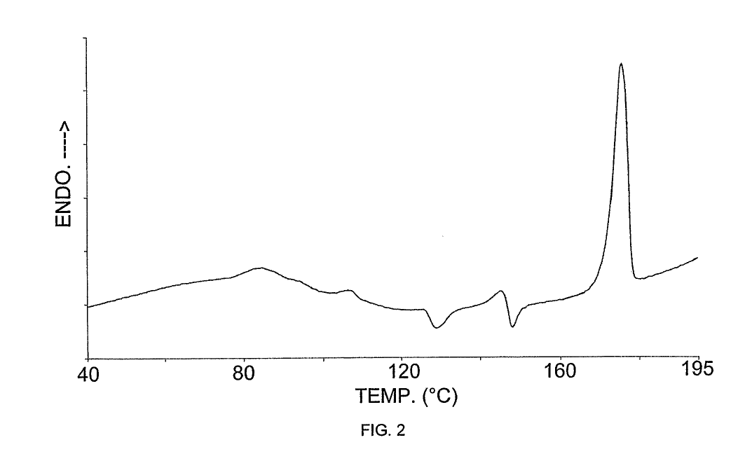

The preferred active for use in the process of the invention is pravastatin sodium, especially crystalline pravastatin sodium, such as the crystalline form defined in U.S. Pat. No. 6,740,775, in particular as defined by the X-ray diffractogram in FIG. 2 of that patent (hereinafter the “LEK” form).

In order to investigate the polymorphic transformations of crystalline pravastatin sodium we have used this crystalline form of pravastatin sodium, which exhibits narrow peaks X-ray diffraction peaks. We first determined that this form of pravastatin sodium does not convert to any other polymorph form when granulated with an alcohol.

We then prepared compositions which were binary mixtures of active pharmaceutical ingredient and one of a selection of excipients. Further we have prepared compositions comprising besides active pharmaceutical ingredient also more than one excipient (functioning as fillers, diluents, binders, disintegrants, lubricants and/or pigment). Certain compositions were coated.

Surprisingly we have discovered that when the active pharmaceutical ingredient was granulated with an alcohol in admixture with microcrystalline cellulose as a diluent a complete or partial conversion of the polymorph could be observed in some instances. The same effect was observed upon granulating the pure pharmaceutical ingredient with alcohol as granulating liquid in which a binder such as polyvinylpyrrolidone was dissolved. The conversion can be observed and measured by detecting and quantitatively assessing the polymorphs present by suitable techniques, for example a suitable thermochemical technique such as DSC or RTG or a suitable spectroscopic technique, such as Raman, IR, XRPD. The DSC method is a cheap, fast, useful and reliable method for determination of the polymorph form of an active pharmaceutical ingredient alone, where one can compare the peaks on the thermograms which may be at different temperatures for different polymorphs, but is also (at least partially) useful for determination of crystalline form of active pharmaceutical ingredient in mixtures with other excipients. However XRPD is the most suitable techniques, since one can accurately measure the relative intensities (area) of peaks specific for each specific polymorph even in mixtures with excipients.

Using wet granulation as one of the steps in the process of preparing pharmaceutical composition comprising pravastatin sodium we have in some instances observed complete or partially transformation into form D, which is contrary to the teaching in WO 01/43723. Having established that an undesired conversion of form Lek to form D can occur during wet granulation, we went on to define the conditions under which no or negligible conversion takes place. The invention lies in stabilizing active pharmaceutical ingredient during the manufacturing of a composition using wet granulation and in a composition during storage and handling by carefully selecting the granulating liquid and its mass ratio to the active as well as carefully selecting the order of addition of filler and/or binder and its mass ratio to the active. We observed that when the ratio of active pharmaceutical ingredient to microcrystalline cellulose is above 1 the polymorphic interconversion problem disappears. We also observed that the ratio of active pharmaceutical ingredient to alcohol is critical, and is ideally above 1 in order to avoid polymorphic interconversions.

Thus, in a preferred embodiment of the invention the weight ratio of active pharmaceutical ingredient to microcrystalline cellulose used in the wet granulation step of preparation of the composition of the invention is at least 1.0, preferably at least 1.25, preferably at least 1.5, optionally at least 2.0. The ratio of active pharmaceutical ingredient to alcoholic liquid in the wet granulation step is preferably at least 1.0, more preferably at least 1.5, more preferably at least 2.0, optionally at least 2.5. The compositions obtained by the process, where those limitations are met are embodiments of our invention.

The alcoholic liquid may be any alcohol or mixture of alcohol with other liquids or solvents, especially aqueous alcoholic solutions. C1-4 alcohols are preferred alcoholic components of the alcoholic liquid, especially ethanol. Absolute ethanol and aqueous ethanolic solutions are the preferred alcoholic liquids used for granulation according to the process of the invention.

Optional fillers may be selected from, powdered cellulose, lactose, starch, pregelatinized starch, sucrose, glucose, mannitol, sorbitol, calcium phosphate, calcium hydrogen phosphate, aluminium silicate, sodium chloride, potassium chloride, calcium carbonate, calcium sulphate, dextrates, dextrin, maltodextrin, glycerol palmitostearate, hydrogenated vegetable oil, kaolin, magnesium carbonate, magnesium oxide, polymethacrylates, talc, and others. Preferred fillers are lactose and cellulose derivatives, such as microcrystalline cellulose.

One of the important excipients used for pharmaceutical composition is a microcrystalline cellulose with average particle size from 10 to approximately 200 microns, preferably 30 to 120 microns, moisture content up to 6%, preferably 1% to 6% with pH from 5 to 7. Due to good plastic deformation qualities of microcrystalline cellulose, pharmaceutical composition comprising it have outstanding mechanical properties such as high breaking strength, high edge strength and low abrasion as well as good disintegration properties, Microcrystalline cellulose is produced by hydrolysis from cellulose, which is comprised of glucose units connected by a 1-4 beta glycosidic bond, the term encompasses any polymer, specifically carbohydrate based polymer, more specifically polymer comprising lactose or glucose units with a high degree of three-dimensional internal bonding resulting in a crystalline structure that is insoluble in water and resistant to reagents, preferably occurring in microfibril structure.

Binders are normally used in the process of manufacturing of a pharmaceutical composition and are generally, where the process comprises a step of preparation of a wet phase often dissolved in a granulation liquid. The binder added in the granulation step may cause the interconversion of the polymorph.

The binder may be starch, pregelatinized starch, gelatin, sodium carboxymethylcellulose, polyvinylpyrrolidone, alginic acid, sodium alginate, acacia, carbomer, dextrin, ehylcellulose, guar gum, hydrogenated vegetable oil, methylcellulose, hydroxyethylcellulose, hydroxypropylcellulose, hydroxypropylmethylcellulose, glucose syrup, magnesium aluminium silicate, maltodextrin, polymethacrylates, zein. Preferred are hydroxypropyl cellulose, hydroxypropyl methylcellulose and most preferred polyvinylpyrrolidone (PVP) which may have different particle size distributions from smaller than 50 microns to larger then 250 microns preferably where 50% of particles lie in the range from 50 to 250 microns, e.g. having a pH of from 3 to 7, e.g. with a water content up to 10%, preferably up to 5% most preferably up to 2.5%, e.g. having a bulk density below 1 g/mL, preferably from 0.3 to 0.7 g/mL and e.g. having average molecular weight from 1000 to 1500000.

When more of the microcrystalline cellulose is used in the preparation of a composition, it should be incorporated in a step different from wet granulation in order to avoid interconversion of polymorphs. Also in case one of the excipients is a binder such as polyvinylpyrrolidone (PVP) it should also be incorporated in a step different from dissolving in a granulating liquid

Methods known in the art can be used to prepare a pharmaceutical composition in accordance with the invention. The stabilized active may be administered in a composition in the form of a powder, pellets, granules, aggregates or any other solid form. The compositions of the present invention include compositions for tableting. The solid dosage forms such as tablets, can be prepared by conventional methods, and are conveniently prepared by wet granulation. In wet granulation at least one of the ingredients can be mixed or contacted with liquid and further processed to provide aggregates, the liquid can be partially or completely removed, by for example drying and optionally more of the same ingredients or other excipients may be further added and solid dosage forms manufactured. Capsules containing the solid composition may be made of gelatin or other encapsulating material.

Pharmaceutical compositions comprising an active pharmaceutical ingredient which exists in a first polymorph form susceptible to conversion into one or more other polymorph forms prepared by a process where at least one of the steps includes preparation of a wet phase can be conveniently produced by first preparing a granulate by spraying a liquid on a dry mixture of the first phase comprising active pharmaceutical ingredient and one or more suitable fillers, binders, disintegrants, glidants, lubricants and other commonly used excipients such as lactose, microcrystalline cellulose, sodium hydrogen phosphate, croscarmelose sodium, sodium lauryl sulfate and povidone. Prepared granulate can be dried, for example in vacuum at 50° C. for up to 5 hours. To the dried and sieved granulate ore or more of further suitable fillers, binders, disintegrants, glidants, lubricants and other commonly used excipients such as lactose, microcrystalline cellulose, croscarmelose sodium, colloidal silicon dioxide, magnesium stearate, aromas, and colors can be added, the mixture blended and homogenized and optionally finished dosage forms such as tablets manufactured on a rotary tableting machine. The liquid can be any conventionally used pharmaceutically acceptable liquid such as alcohols, such C1-C4 alcohols (i.e. methanol or ethanol), ketone (i.e. acetone), and water, or mixtures thereof, preferably alcoholic liquid that is one comprising ethanol or methanol.

Compositions such as tablets, granulates and powders may be coated. The coatings may comprise hydropropylmethylcellulose, hydroxypropylcellulose, hydroxyethylcellulose, methylcellulose, polyvinylpyrrolidone, ethylcellulose, methacrylate polymers and methacrylate/trimethylammonioethylmethacrylate copolymers (e.g., different grades of Eudragit), phthalic acid cellulose acetate, hydroxypropylmethyl cellulose phthalate, polyvinyl alcohol phthalate, carboxymethylethylcellulose, a copolymer of styrene and maleic acid, a copolymer of methacrylic acid and methyl methacrylate, and like materials, or any combination of the polymers and if desired, they may be employed with suitable excipients such as plasticizers and/or extending agents or others. A coated tablet may have a coating on the surface of the tablet or may be a tablet comprising a coated powder or coated granules.

The present invention is especially embodied in the method of stabilizing crystalline pravastatin sodium present in polymorph form characterized by an X-ray diffraction pattern exhibiting narrow peaks in the composition comprising microcrystalline cellulose which can any one of commonly available materials such as Avicel produced by FMC or Microcel produced by Blanver or Vivapur produced by J. Rettenmaier & Sohne, or any other microcrystalline cellulose or equivalent material.

The specific embodiment of our invention is a method of stabilizing in a pharmaceutical composition an active pharmaceutical ingredient which exists in a first polymorph form susceptible to conversion into one or more other polymorph forms, where the excipients comprise microcrystalline cellulose and a liquid is used in preparation of aforesaid pharmaceutical composition, characterized in that the ratio of active pharmaceutical ingredient and microcrystalline cellulose in preparation of wet phase used in preparation of aforesaid pharmaceutical composition is above 1 and the ratio of active pharmaceutical ingredient and alcohol used in preparation of wet phase used in preparation of aforesaid pharmaceutical composition is above 1 and a process for stabilizing such composition. Specifically where wet phase is an alcoholic phase (the liquid used is an alcoholic liquid) preferably consisting only absolute ethanol or of an aqueous ethanol solution.

More specifically the invention is embodied in a process as described wherein weight ratio of active pharmaceutical ingredient to the liquid is above 2.0.

The object of our invention are also the processes for preparing a composition where microcrystalline cellulose is incorporated into the composition in more than one step.

Generally the invention is embodied in a process whereby pravastatin sodium in a first polymorph form is stabilized against conversion into a polymorph form which exhibits broad peaks in X-ray diffraction pattern, having half-value widths of significant peaks above 2° 2θ.

More specifically the embodiment of the invention is a process as described wherein the active pharmaceutical ingredient is pravastatin sodium, specifically having characteristic peaks in a X-ray diffractogram at 2θ of 4, 10.2, 16.3, 17.3, and 20.0±0.20 that is exhibiting an X-ray diffraction pattern substantially similar to that in FIG. 2 of U.S. Pat. No. 6,740,775. and the weight ratio of pravastatin sodium to microcrystalline cellulose is above 1.0 and the weight ratio of pravastatin sodium to ethanol is above 2.0.

The embodiments of the invention are also the product obtainable by above described process especially pharmaceutical compositions. Pharmaceutical compositions may besides one or more active ingredients comprise inactive ingredient, among them one or more binders. In an embodiment of the invention where a binder is incorporated into a composition this should performed in a step other than the step of preparation of an alcoholic phase, especially where binder is polyvinylpyrrolidone (PVP).

The X-ray diffraction pattern of a first polymorphic form is considered substantially similar to that of the second form, when it comprises the characteristic peaks of the second form and the 2θ values of each of those peaks lie within ±0.2°, preferably ±0.1° of the 2θ values of the characteristic peaks of the second form. The characteristic peaks are those exhibiting the highest intensity at the number of measurements. Thus, for instance, an X-ray diffraction pattern of pravastatin sodium comprising peaks at 4, 10.2, 16.3, 17.3, and 20.0±0.2° 2θ is considered substantially similar to that of form LEK.

### EXAMPLES

The examples are provided for illustrative purposes only, and are not intended to limit the invention in any way.

The following Examples show the extent of conversion of an active pharmaceutical ingredient which exists in a first polymorph form into one or more other polymorph forms.

### Differential Scanning Calorimetry (DSC):

Samples are measured on apparatus Perkin-Elmer Analytical Instruments Pyris 1 DSC. Mass of the samples is 1.5 mg; samples are thermal balanced for 1 minute at 30° C. and then heated from 30 to 200° C. at 10 K/min.

### X-Ray Powder Diffraction (XRPD) Analysis:

References to 2θ values are to those measured using CuKα radiation. Samples are measured on apparatus Siemens D-5000 by reflex technique at two conditions:
a. Samples with high amount of pravastatin sodium (more than 30%): CuKα radiation, range from 2° to 37° 2θ, step 0.04° 2θ, integration time 1 second, slots V20 and 0.6 mm.
b. Samples with low amount of pravastatin sodium (less than 30%): CuKα radiation, range from 3° to 12° 2θ, step 0.04° 2θ, integration time 15 second, slots V20 and 0.6 mm.

### Example 1

15 g of pravastatin sodium are added to a vessel and while mixing 15 g of ethanol is sprayed onto the sample. The granules thus formed are dried under vacuum at room temperature for 12 hours. The dry sample is analyzed with XRPD and DSC. The sample contains crystalline pravastatin sodium form LEK, confirmed by both techniques.

### Example 2

12.4 g of pravastatin sodium are added to a vessel and while mixing 12 g of a 20% solution of PVP K25 in ethanol is sprayed onto the sample. The granules thus formed are dried under vacuum at room temperature for 12 hours. The dry sample is analyzed with XRPD and DSC. The sample contains crystalline pravastatin sodium form LEK and a small amount of form D, confirmed by XRPD.

### Example 3

14.8 g of pravastatin sodium is added to a vessel and while mixing 9 g of a 6.3% solution of water in ethanol is sprayed onto the sample. The granules thus formed are dried under vacuum at 50° C. for 12 hours. The dry sample is analyzed with XRPD. The sample contains crystalline pravastatin sodium form LEK.

### Example 4

9.9 g of pravastatin sodium is added to a vessel and while mixing 9 g of a solution of PVP K25 (20%) and water (4.4%) in ethanol is sprayed onto the sample. The granules thus formed are dried under vacuum at 50° C. for 12 hours. The dry sample is analyzed with XRPD. The sample contains crystalline pravastatin sodium form LEK.

The results of Examples 1-4 are summarized in Table I

TABLE 1
Polymorph analysis results after granulation of crystalline pravastatin
sodium form LEK with ethanol and ethanol solution of PVP

| Example | | XRPD | DSC |
|---|---|---|---|
| No. | Experiment conditions | results | results |
| Error! | 15 g pravastatin Na + 15 g ethanol, | form LEK | form LEK |
| Reference | drying in vacuum at RT, 12 h | | |
| source | | | |
| not | | | |
| found. | | | |
| Error! | 12.4 g pravastatin Na + 12 g of | form LEK + | — |
| Reference | 20% PVP solution in ethanol, | form D | |
| source | drying in vacuum at RT, 12 h | | |
| not | | | |
| found. | | | |
| Error! | 14.8 g pravastatin Na + 9 g of | form LEK | — |
| Reference | ethanol containing 6.3% water, | | |
| source | drying in vacuum at 50° C., 12 h | | |
| not | | | |
| found. | | | |
| Error! | 9.9 g pravastatin Na + 9 g of 20% | form LEK | |
| Reference | PVP solution in wet ethanol | | |
| source | (4.4% water), drying in vacuum | | |
| not | at 50° C., 12 h | | |
| found. | | | |

These Examples demonstrate that use of alcohol as a granulating liquid, for example absolute ethanol or aqueous ethanol, does not cause the precrystallization (conversion into another polymorph form) of pravastatin sodium in the absence of other ingredients. However, granulation with a granulating liquid comprising a binder (PVP) does in some experiments induce a partial conversion as summarized in Table 1. (You need to add an explanation here or later of what is special about the conditions under which PVP does, or does not, induce precrystallization)

The following Examples demonstrate the influence of granulating liquid (ethanol, water) optionally comprising polyvinylpyrrolidone on the one hand, and the influence of additional excipients in certain weight ratios on the other hand (microcrystalline cellulose, lactose, anhydrous disodium hydrogenphosphate, crosslinked carboxymethylcellulose sodium and sodium lauryl sulfate) on the interconversion of an active pharmaceutical ingredient which exists in a first polymorph into one or more other polymorph forms.

Avicel™, Vivapuir™ and Microcel™ are commercially available forms of microcrystalline cellulose.

### Example 5

3 g of pravastatin sodium and 12.6 g of Avicel PH 112 are added to a vessel and while mixing 10 g of ethanol is sprayed onto the sample. A portion of the granules thus formed are dried under vacuum at room temperature and the remainder at 50° C. for 12 hours. Both dried samples are analyzed with XRPD. They both contain pravastatin sodium in form D.

### Example 6

3 g of pravastatin sodium and 12 g of dried Avicel PH 112 are added to a vessel and while mixing 10 g of ethanol is sprayed onto the sample. A portion of the granules thus formed are dried under vacuum at room temperature and the remainder at 50° C. for 12 hours. Both dried samples are analyzed using XRPD. They both contain pravastatin sodium in form D.

### Example 7

6 g of pravastatin sodium and 3 g of Lactose 80 mesh are added to a vessel and while mixing 9 g of ethanol is sprayed onto the sample. The granules thus formed are dried under vacuum at 50° C. for 12 hours. The dry sample is analyzed with XRPD. The sample contains crystalline pravastatin sodium form LEK.

### Example 8

5 g of pravastatin sodium and 6 g of anhydrous disodium hydrogenphosphate are added to a vessel and while mixing 9 g of ethanol is sprayed onto the sample. The granules thus formed are dried under vacuum at 50° C. for 12 hours. The dry sample is analyzed with XRPD. The sample contains crystalline pravastatin sodium form LEK.

### Example 9

10 g of pravastatin sodium and 2 g of Ac-Di-Sol are added to a vessel and while mixing 11 g of ethanol is sprayed onto the sample. The granules thus formed are dried under vacuum at 50° C. for 12 hours. The dry sample is analyzed with XRPD. The sample contains crystalline pravastatin sodium form LEK.

### Example 10

10 g of pravastatin sodium and 2 g of Texapon™ (sodium lauryl sulfate) are added to a vessel and while mixing 11 g of ethanol is sprayed onto the sample. The granules thus formed are dried under vacuum at 50° C. for 12 hours. The dry sample is analyzed with XRPD. The sample contains crystalline pravastatin sodium form LEK.

### Example 11

4 g of pravastatin sodium and 2 g of Avicel PH 112 are added to a vessel and while mixing 9 g of ethanol is sprayed onto the sample. The granules thus formed are dried under vacuum at 50° C. for 12 hours. The dry sample is analyzed with XRPD. The sample contains a mixture of crystalline pravastatin sodium form LEK and form D.

### Example 12

4 g of pravastatin sodium and 2 g of Avicel PH 112 are added to a vessel and while mixing 3 g of ethanol is sprayed onto the sample. The granules thus formed are dried under vacuum at 50° C. for 12 hours. The dry sample is analyzed with XRPD. The sample contains crystalline pravastatin sodium form LEK.

### Example 13

6 g of pravastatin sodium and 6 g of Avicel PH 112 are added to a vessel and while mixing 3 g of ethanol is sprayed onto the sample. The granules thus formed are dried under vacuum at room temperature for 12 hours. The dry sample is analyzed with XRPD. The sample contains crystalline pravastatin sodium form LEK.

### Example 14

6 g of pravastatin sodium and 6 g of Vivapur 103 are added to a vessel and while mixing 3 g of ethanol is sprayed onto the sample. The granules thus formed are dried under vacuum at room temperature for 12 hours. The dry sample is analyzed with XRPD. The sample contains crystalline pravastatin sodium form LEK.

### Example 15

6 g of pravastatin sodium and 6 g of Microcel are added to a vessel and while mixing 3 g of ethanol is sprayed onto the sample. The granules thus formed are dried under vacuum at room temperature for 12 hours. The dry sample is analyzed with XRPD. The sample contains crystalline pravastatin sodium form LEK.

### Example 16

6 g of pravastatin sodium and 6 g of Avicel PH 112 are added to a vessel and while mixing 7 g of ethanol is sprayed onto the sample. The granules thus formed are dried under vacuum at room temperature for 12 hours. The dry sample is analyzed with XRPD. The sample contains crystalline pravastatin sodium form LEK and small amount of form D.

### Example 17

0.5 g of pravastatin sodium and 0.5 g of Avicel PH 112 are added to a vessel and homogenized. Dry mixture is exposed to 60° C. for 2 hours. The sample is analyzed with XRPD and it contains crystalline pravastatin sodium form LEK.

TABLE 2
Polymorph analysis results of granulation of crystalline pravastatin sodium
form LEK together with excipients using ethanol as a granulating liquid

| Example | | XRPD |
|---|---|---|
| No. | Experiment conditions | results |
| Error! | 12.6 g Avicel + 3 g pravastatin Na + 10 g | form D |
| Reference | ethanol, drying in vacuum at RT and 50° C. | |
| source not | | |
| found. | | |
| Error! | 12 g dried Avicel + 3 g pravastatin Na + 10 g | form D |
| Reference | ethanol, drying in vacuum at RT and 50° C. | |
| source not | | |
| found. | | |
| Error! | 3 g lactose + 6 g pravastatin Na + 9 g ethanol, | form LEK |
| Reference | drying in vacuum at 50° C. | |
| source not | | |
| found. | | |
| Error! | 6 g Na₂HPO₄ + 5 g pravastatin Na + 9 g ethanol, | form LEK |
| Reference | drying in vacuum at 50° C. | |
| source not | | |
| found. | | |
| Error! | 2 g Ac-Di-Sol + 10 g pravastatin Na + 11 g | form LEK |
| Reference | ethanol, drying in vacuum at 50° C. | |
| source not | | |
| found. | | |
| Error! | 1 g Texapon + 10 g pravastatin Na + 11 g | form LEK |
| Reference | ethanol, drying in vacuum at 50° C. | |
| source not | | |
| found. | | |
| Error! | 2 g Avicel + 4 g pravastatin Na + 9 g ethanol, | form |
| Reference | drying in vacuum at 50° C. | LEK + |
| source not | | form D |
| found. | | |
| Error! | 2 g Avicel + 4 g pravastatin Na + 3 g ethanol, | form LEK |
| Reference | drying in vacuum at 50° C. | |
| source not | | |
| found. | | |
| Error! | 6 g Avicel + 6 g pravastatin Na + 3 g ethanol, | form LEK |
| Reference | drying in vacuum at RT | |
| source not | | |
| found. | | |
| Error! | 6 g Vivapur + 6 g pravastatin Na + 3 g ethanol, | form LEK |
| Reference | drying in vacuum at RT | |
| source not | | |
| found. | | |
| Error! | 6 g Microcel + 6 g pravastatin Na + 3 g ethanol, | form LEK |
| Reference | drying in vacuum at RT | |
| source not | | |
| found. | | |
| Error! | 6 g Avicel + 6 g pravastatin Na + 7 g ethanol, | form |
| Reference | drying in vacuum at RT | LEK + |
| source not | | form D |
| found. | | |
| Error! | 0.5 g Avicel + 0.5 g pravastatin Na, dry mixture, | form LEK |
| Reference | 2 h on 60° C. | |
| source not | | |
| found. | | |

These Examples show that conversion of the polymorph form is detected when microcrystalline cellulose such as Avicel™, Vivapur™ or Microcel™ is used at certain ratios to active pharmaceutical ingredient, and this phenomenon is also dependent on the amount of granulating liquid used.

One can conclude that pravastatin sodium precrystallizes to form D in the presence of a high amount of microcrystalline cellulose and granulating liquid. Pravastatin sodium in the Lek form is, however, stable if the mass ratio of pravastatin sodium to microcrystalline cellulose is higher or equal to 1:1 and mass ratio of pravastatin sodium to ethanol is higher or equal to 1:1, but preferably 1:0.5.

### Example 18

Granulate comprising pravastatin sodium is prepared as follows: First phase of granulate contains: 60 g Pravastatin sodium (form LEK), 30 g Avicel PH 112, 30 g Lactose 80 mesh, 2 g anhydrous disodium hydrogenphosphate, 10.8 g Ac-Di-Sol, 3.0 g Texapon, and 18 g polyvinylpyrrolidone K25. While mixing, 23.7 g of ethanol is sprayed onto the dry mixture of above first phase. The granules thus formed are dried under vacuum at 50° C. for 5 hours. To the dried and sieved granulate further components are added: 0.45 g brown iron oxide, 359 g Avicel PH 112, 10.8 g Ac-Di-Sol, 3.0 g Aerosil 200, 3.0 g magnesium stearate. The mixture is blended and homogenized. The first phase of granulate (after granulation and drying) is analyzed with XRPD and no conversion of polymorph form detected.

### Example 19

0.4 g of granulate sample of the first phase from the previous experiment is stored in glass vials, whereby part of the samples are moistened (1.25% of water) and the remainder kept dry. Hermetically closed vials are exposed to a temperature of 60° C. and samples analyzed with XRPD after 1, 3, 7 and 14 days of storage at this temperature. Neither sample of pravastatin sodium is found to precrystallize. Tablets prepared according to the composition of the previous Example have been subjected to accelerated stability testing at 60° C. for one month, confirming stability.

In order to prepare granulates for tableting the amount of ethanol needed to ensure appropriate properties in the wet granulate is proportional to the amount of microcrystalline cellulose added into the first phase. Thus, when only a small portion of the total microcrystalline cellulose is added into the first phase of granulate (ratio pravastatin sodium:microcrystalline cellulose=1:0.5) the quantity of ethanol needed for wet granulation is low and the mass ratio of pravastatin sodium and ethanol is 1:0.4. Under those conditions no precrystallization occurs.

Other excipients (lactose, anhydrous disodium hydrogenphosphate, crosslinked carboxy-methylcellulose sodium (Ac-Di-Sol) and sodium lauryl sulfate (Texapon)) and the drying temperature do not influence the extent of recrystallization of pravastatin sodium during wet granulation with ethanol. Also the exposure of a dry mixture of pravastatin sodium and microcrystalline cellulose for 2 hours at 60° C. does not cause any precrystallization.

1. A process for the preparation of a pharmaceutical composition comprising an active pharmaceutical ingredient capable of existing in multiple polymorphic forms, comprising a step of preparation of a wet phase comprising said active pharmaceutical ingredient and microcrystalline cellulose and a liquid, wherein in said wet phase has a weight ratio of active pharmaceutical ingredient to microcrystalline cellulose above 1.0 a weight ratio of active pharmaceutical ingredient to liquid above 1.0.

2. A process according to claim 1 wherein said wet phase is an alcoholic phase and in said wet phase the weight ratio of active pharmaceutical ingredient to microcrystalline cellulose is above 1.0 and the weight ratio of active pharmaceutical ingredient to alcoholic liquid is above 1.0.

3. A process according to claim 1 wherein said weight ratio of active pharmaceutical ingredient to the liquid is above 2.0.

4. A process according to claim 1 wherein said liquid is an alcoholic liquid consisting of only absolute ethanol or of an aqueous ethanol solution.

5. A process according to claim 1 wherein said microcrystalline cellulose is incorporated into the composition in more than one step.

6. A process according to claim 1 wherein the active pharmaceutical ingredient is pravastatin sodium.

7. A process according to claim 6 wherein the liquid is ethanol and the weight ratio of pravastatin sodium to microcrystalline cellulose is above 1.0 and the weight ratio of pravastatin sodium to ethanol is above 2.0.

8. A process according to claim 1 wherein the active pharmaceutical ingredient is crystalline pravastatin sodium having characteristic peaks in a X-ray diffractogram at 2θ of 4, 10.2, 16.3, 17.3, and 20.0±0.2°.

9. A process according to claim 8 wherein the crystalline pravastatin sodium exhibits an X-ray diffraction pattern substantially similar to that in FIG. 2 of U.S. Pat. No. 6,740,775.

10. A process according to claim 6 whereby pravastatin sodium in a first polymorph form is stabilized against conversion into a polymorph form which exhibits broad peaks in X-ray diffraction pattern, having half-value widths of significant peaks above 2° 2 Theta.

11. A process according to claim 1 wherein a binder is incorporated into the composition in a step other than the step of preparation of an alcoholic phase.

12. A process according to claim 11 wherein said binder is polyvinylpyrrolidone (PVP).

13. A pharmaceutical composition obtainable by the process of claim 1.

14. A stabilized pharmaceutical composition comprising the polymorph form of pravastatin sodium which exhibits X-Ray diffraction pattern with significant peaks having half-value widths below 2° 2 Theta characterized in that the polymorph form of pravastatin sodium is stabilized against converting into one exhibiting peaks in X-ray diffraction pattern, having half-value widths of significant peaks above 2° 2 Theta.

15. A method of using the pharmaceutical composition according to claim 13 for the manufacture of a medicament for the treatment of hypercholesterolemia.

16. A method of preventing or treating hypercholesterolemia in a susceptible patient, comprising administering to said patient a therapeutically effective amount of the pharmaceutical composition of claim 13.
